(19) United States
(12) Patent Application Publication
(10) Pub. No.: US 2004/0152684 A1
(43) Pub. Date: Aug. 5, 2004

(54) Use of antigestagens for inhibiting accelerated endometrial maturation during infertility treatment

(76) Inventors: Christa Hegele-Hartung, Muelheim a.d Ruhr (DE); Holger Hess-Stumpp, Berlin (DE); Henning M. Beier, Aachen (DE); Claudia Krusche, Aachen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/747,060

(22) Filed: Dec. 30, 2003

Related U.S. Application Data
(63) Continuation of application No. 09/801,925, filed on Mar. 9, 2001, now abandoned.
Application No. 09/801,925 is a continuation-in-part of application No. 09/756,286, filed on Jan. 9, 2001, now abandoned.

(51) Int. Cl. A61K 31/56

(52) U.S. Cl. 514/177

(57) ABSTRACT

A method of inhibiting the occurrence of advanced endometrium maturation in a female mammal undergoing fertility treatment is disclosed. The method comprises the administration at least one 17α-fluoralkylated progesterone receptor antagonist to the female mammal.

This application is a continuation in part of U.S. Ser. No. 09/756,286 filed Jan. 9, 2001, which is incorporated by reference herein in its entirety.

### BACKGROUND OF THE INVENTION

This invention relates to the use of antigestagens for shifting post-ovulatory endometrial maturation during infertility treatment.

Ovarian stimulation with gonadotropins is commonly used in humans in assisted reproductive technologies, including in vitro fertilization and embryo transfer therapy (IVF/ET). However, the post-ovulatory endometrial transformation is advanced after controlled ovarian hyperstimulation (Garcia et al., 1984, Fertil. Steril. 41:31-37; Paulson et al., 1997, Fertil. Steril. 76:321-325; Kolb, 1997, Fertil. Steril. 67:625-630; Franchin et al., 1999, Fertil. Steril. 71:174-181.) As a consequence, the usual precisely synchronized temporal development of the endometrium and the embryo is disrupted, resulting in low implantation rates for healthy blastocysts.

### SUMMARY OF THE INVENTION

This invention provides a method of enhancing fertility in mammals by the administration of 17α-fluoralkylated progesterone receptor antagonists to inhibit the advancement of endometrium maturation during infertility (i.e., fertility enhancement) treatment including all assisted reproduction therapies.

It is also contemplated as part of this invention that such a method will be particularly useful in a female mammal undergoing fertility treatment. Fertility treatment, as used herein, refers to any treatment which is rendered to a female mammal for the purposes of achieving a pregnancy, whether or not the mammal has been determined to be fertile, infertile, or to have impaired fertility. Fertility treatments contemplated as part of this invention include, but are not limited to ovarian hyperstimulation (OH), in vitro fertilization and embryo transfer (IVF/ET), and OH in combination with IVF/ET. Particularly, a method of inhibiting advanced endometrial maturation, which typically occurs in conjunction with ovarian hyperstimulation, is contemplated. For example, ovarian hyperstimulation may be used as a tool in fertility treatment in conjunction with, for example, timed intercourse, artificial insemination, intrauterine insemination, IVF/ET, and gamete intra fallopian transfer (GIFT).

IVF/ET, as used herein, refers to any techniques wherein oocytes are fertilized in vitro and then transferred into the female reproductive tract. For example, transfer of the embryo may occur via transfer of the embryo into the uterus through the cervix or via transfer of the embryo into the fallopian tubes (zygote intra fallopian transfer (ZIFT)). Fertilization of oocytes in vitro may be accomplished, for example, by incubating oocytes in the presence of sperm or by intra cytoplasmic sperm injection (ICSI). IVF/ET may also be accomplished using donor eggs. Infertility treatments also include treatments such as induction of ovarian stimulation followed by fertilization by normal coitus.

Ovarian hyperstimulation, as used herein, refers to the use of a follicle stimulating agent to stimulate follicle development. Ovarian hyperstimulation may be used in female subjects having a variety of ovulatory conditions, including subjects who are anovulatory, subjects who have impaired, reduced, or irregular ovulation, and subjects with normal ovulatory patterns. Preferred follicle stimulating agents, include gonadotropins, such as for example, follicle stimulating hormone (FSH), luteinizing hormone (LH), human menopausal gonadotropins (hMG), and human chorionic gonadotropin (hCG). Brand names of gonadotropins that are available for use as follicle stimulating agents, include for example, Perganol, Metrodin, Humegon, Fertinorm, Gonal F, and Primogonyl-1000 etc. Follicle stimulating agents, as used herein, also include estrogen blocking agents such as, for example, clomiphene citrate (commercially available as Clomid and Serophene). Gonadotropins that may be used in accordance with this invention include hormones that are isolated from naturally occurring source materials and hormones that are produced synthetically, including hormones produced through recombinant DNA techniques. Mutant gonadotropins which retain activity as follicle stimulating agents are also contemplated as part of this invention.

Ovarian hyperstimulation may be accomplished using more than type of follicular stimulating agent. For example, an estrogen blocking agent such as clomiphene may be used in combination with a gonadotropin. Ovarian hyperstimulation may used to treat a variety of types of infertility, including but not limited to, idiopathic infertility, anovulatory infertility, endometriosis associated infertility, tubal factor infertility and male factor infertility.

Ovarian hyperstimulation may also be accomplished in conjunction with a gonadotropin releasing hormone antagonist (GnRH) to turn off the subjects endogenous hormone production. GnRH's that may be used in conjunction with the invention include, for example, Synarel or Lupron.

In a preferred embodiment, the methods of this invention are employed in conjunction with human female subjects. The methods of this invention may also be employed with other mammalian female subjects, including but not limited to, household pets, farm animals and zoo animals, particularly including cows, pigs, horses, and sheep. When used in fertility treatment of non-human mammals, the administration of antigestagens can help to achieve a higher success rate in, for example, in vitro fertilization and embryo transfer undertaken for economic or breeding purposes.

Non-human mammals which are produced by the methods disclosed herein are also contemplated as part of the invention. These non-human mammals include, for example, mammals which have been genetically modified, including for example, mammals that have been genetically modified using recombinant DNA techniques.

In one aspect, the invention relates to a method of inhibiting the occurrence of advanced endometrium maturation in a human female subject undergoing fertility treatment comprising administering at least one 17α-fluoralkylated progesterone receptor antagonist to the female subject during the post-ovulatory phase of the endometrial cycle.

In another aspect, the invention relates to a method of achieving pregnancy in a human female subject comprising stimulating the ovaries of the subject by administering a follicle stimulating agent to the subject, wherein the agent comprises follicle stimulating hormone; removing eggs from the ovary of the stimulated subject; administering at least one 17α-fluoralkylated progesterone receptor antagonist to the subject in the post-ovulatory phase of the endometrial cycle; fertilizing at least one egg in vitro to obtain an embryo; implanting the embryo in the uterus or fallopian tubes of the mammal.

In another aspect, the invention relates to a method of inhibiting the occurrence of advanced endometrium maturation in a non-human female mammal undergoing fertility treatment to achieve pregnancy comprising administering at least one 17α-fluoralkylated progesterone receptor antagonist to the mammal during the post-ovulatory phase of the endometrial cycle.

In another aspect, the invention relates to a method of achieving pregnancy in a non-human mammal comprising stimulating the ovaries of the mammal by administering a follicle stimulating agent to the mammal, wherein the agent comprises follicle stimulating hormone; removing eggs from the ovary of the stimulated mammal; administering at least one 17α-fluoralkylated progesterone receptor antagonist to the mammal in the post-ovulatory phase of the endometrial cycle; fertilizing at least one egg in vitro to obtain an embryo; implanting the embryo in the uterus or fallopian tubes of the mammal.

In a further aspect, the invention relates to a method of achieving pregnancy in a female mammal comprising

(a) administering gonadotropins for ovarian hyperstimulation to the mammal;

(b) removing eggs from the ovary of the stimulated animal;

(c) administering at least one 17α-fluoralkylated progesterone receptor antagonist;

(d) fertilizing at least one of the removed eggs in vitro to obtain an embryo

(e) introducing the embryo into the reproductive tract of the mammal.

Preferably, the gonadotropin administered to the mammal to achieve ovarian hyperstimulation comprises follicle stimulating hormone (FSH). Even more preferably ovarian stimulation is achieved by the use of a first gonadotropin comprising FSH and the subsequent use of second gonadotropin comprising chorionic gonadotropin, particularly human chorionic gonadotropin (hCG). Preferably the embryo is introduced into the reproductive tract of the mammal by introduction into the uterus via the cervix. Also preferably, a gonadotropin releasing hormone (GnRH) agonist or antagonist is administered to the female mammal prior and during the administration of the gonadotropins.

Compounds useful as antigestagens according to the present invention include all 17α-fluoralkylated progesterone receptor antagonists which possess a strong affinity for the gestagen receptor (progesterone receptor) and show minimal gestagen activity of their own. For example, 17α-fluoralkylated steroidal compounds which may be employed in conjunction with the invention are described U.S. patent application Ser. No. 09/020,947, WO/98/34947, Wang and Ruan, 1994, Journal of Fluorine Chemistry 69:1-3, all of which are hereby incorporated by reference in their entirety. Antigestagens useful for the present invention include but are not limited to, for example, 17α-fluoroalkyl steroids of general formula I, primarily from the cited disclosures:

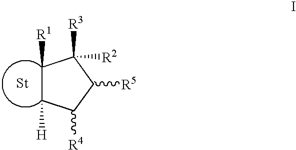

wherein

R¹ is methyl or ethyl,

R² is CₙFₘHₒ, wherein n is 1-6, preferably 2, 3, 4, 5 or 6, m>1 and m+o=2n+1,

R³ is a free, etherified or esterified hydroxy group,

R⁴ and R⁵ each is a hydrogen, or together form an additional bond or a methylene group,

St is a steroidal ABC-ring system of partial formula A, B or C

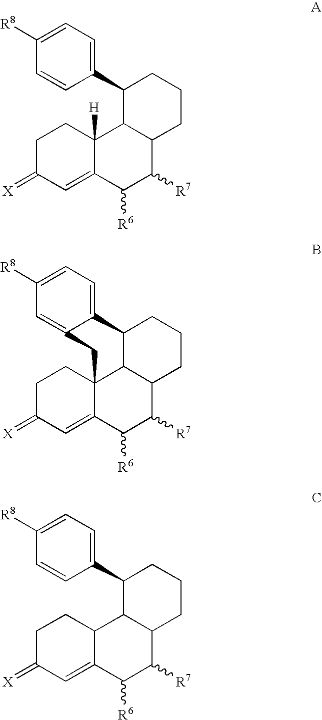

wherein

R⁶ is hydrogen, a straight-chain C₁-C₄ alkyl group or branched C₃-C₄ alkyl group or halogen,

R⁷ is hydrogen, a straight-chain C₁-C₄ alkyl group or a branched C₃-C₄ alkyl group, or

if St is a steroidal ABC-ring system A or B, in addition

R⁶ and R⁷ together can form an additional bond,

X is oxygen, hydroxyimino (═N—OH) or two hydrogen atoms,

R⁸ is Y or aryl that is optionally substituted in several places with a group Y, other than H,

Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,

R^{9a} and R^{9b} are the same or different and each is hydrogen or C₁-C₁₀ alkyl,

R⁹ is hydrogen or C₁-C₁₀ alkyl,

and for —NR^{9a}R^{9b} radicals, as well as their physiologically compatible salts with acids and for —CO₂R⁹ radicals with R⁹ being hydrogen, as well as their physiologically compatible salts with bases.

The wavy lines mean that the substituent in question can be in α- or β-position.

Suitable alkyl groups within the scope of this formula include methyl, ethyl, or n- or iso-propyl, and n-, iso- or tert-butyl groups.

The other C₁-C₁₀ alkyl groups, Y, R⁹, R^{9a}, R^{9b}, include the higher homologs in addition, such as, for example, the pentyl, neo-pentyl, and hexyl to decyl groups.

C₁-C₁₀ alkyl groups are to be understood to encompass, however, carbocyclic or alkylcycloalkyl groups as well with up to 10 carbon atoms, for example cyclopropyl, cyclopentyl, cycloheptyl, methylcyclopropyl, methylcyclopentyl or methylcyclohexyl. A methyl or ethyl group is preferred for all cases above.

C₁-C₁₀ alkoxy groups are the radicals that are lengthened by one oxygen atom and derived from the alkyl groups that are mentioned above, thus, e.g., the methoxy, ethoxy, n- or iso-propoxy, n-, iso- or tert-butoxy radical, etc.

C₁-C₁₀ alkanoyl is defined as the acyl radicals of straight-chain and branched C₁-C₁₀ alkanecarboxylic acids, thus, for example, the formyl, acetyl, propionyl, butyryl or iso-butyryl radical, etc.

C₁-C₁₀ alkanoyloxy radicals are the radicals of the above alkanoyl radicals that are lengthened by one oxygen atom, thus, e.g., the acetyloxy, propionyloxy, and butyryloxy, etc.

If a halogen atom is mentioned as a substituent, this can be a fluorine, chlorine or bromine atom. Fluorine is preferred.

For radicals R², perfluorinated side chains of length n=2-4 are preferred and among the latter, in turn the pentafluoroethyl unit is especially preferred.

R³ stands primarily for a free hydroxy group.

In the case of an etherified or esterified hydroxy group as a 17β-substituent, the latter is preferably etherified with a C₁-C₁₀ alkyl group or esterified with a C₁-C₁₀ alkanoyl group. For this alkyl or alkanoyl group, the same meanings as above apply. The etherification or esterification of the hydroxy group is carried out according to the methods that are familiar to one skilled in the art.

R⁴ and R⁵ preferably each are a hydrogen or together form an additional bond.

If R⁸ is a group Y, this is preferably a C₁-C₁₀ alkanoyl or (1-hydroxy)-C₁-C₁₀ alkyl group; among these, acetyl and propionyl are especially preferred.

Preferred carbocyclic or heterocyclic aryl radicals are phenyl, 1- or 2-naphthalinyl, 2- or 3-furanyl, 2- or 3-benzofuranyl, 2- or 3-thienyl, 2-, 3- or 4-pyridinyl. Substituted aryl radicals R⁸, are primarily 4-cyanophenyl and 4-halophenyl, especially 4-fluorophenyl, can be cited.

Among all the radicals that are mentioned as preferred for R⁸, R⁸ in the meaning of Y and Y, in turn, equal to acetyl is especially to be preferred.

The compounds that are mentioned below are especially preferred according to the invention:

11β-(4-Acetylphenyl)-17β-hydroxy-17α-(1,1,2,2,2-pentafluoroethyl)estr-4-en-3-one;

4′-[17β-hydroxy-3-oxo-17α-(1,1,2,2,2-pentafluoroethyl)estr-4-en-11β-yl][1,1′-biphenyl]-4-carbonitrile;

11β-(4′-fluoro[1,1′-biphenyl]-4-yl)-17β-hydroxy-17α-(1,1,2,2,2-pentafluoroethyl)estr-4-en-3-one;

17β-hydroxy-17α-(1,1,2,2,2-pentafluoroethyl)-11β-[4-(3-pyridinyl)phenyl]estr-4-en-3-one;

11β-(4-acetylphenyl)-17β-hydroxy-17α-(1,1,2,2,2-pentafluoroethyl)estra-4,15-dien-3-one;

4′-[17β-hydroxy-3-oxo-17α-(1,1,2,2,2-pentafluoroethyl)estra-4,15-dien-11β-yl][1,1′-biphenyl]-4-carbonitrile;

11β-(4′-fluoro[1,1′-biphenyl]-4-yl)-17β-hydroxy-17α-(1,1,2,2,2-pentafluoroethyl)estra-4,15-dien-3-one;

17β-hydroxy-17α-(1,1,2,2,2-pentafluoroethyl)-11β-[4-(3-pyridinyl)phenyl]estr-4,15-dien-3-one;

11β-(4-acetylphenyl)-17β-hydroxy-17α-(1,1,2,2,2-pentafluoroethyl)estra-4,9-dien-3-one;

4′-[17β-hydroxy-3-oxo-17α-(1,1,2,2,2-pentafluoroethyl)estra-4,9-dien-11β-yl][1,1′-biphenyl]-4-carbonitrile;

11β-(4′-fluoro[1,1′-biphenyl]-4-yl)-17β-hydroxy-17α-(1,1,2,2,2-pentafluoroethyl)estra-4,9-dien-3-one;

17β-hydroxy-17α-(1,1,2,2,2-pentafluoroethyl)-11β-[4-(3-pyridinyl)phenyl]estra-4,9-dien-3-one;

11β-(4-acetylphenyl)-17β-hydroxy-17α-(1,1,2,2,2-pentafluoroethyl)estra-4,9,15-trien-3-one;

4′-[17β-hydroxy-3-oxo-17α-(1,1,2,2,2-pentafluoroethyl)estra-4,9,15-trien-11β-yl][1,1′-biphenyl]4-carbonitrile;

11β-(4′-fluoro[1,1′-biphenyl]-4-yl)-17β-hydroxy-17α-(1,1,2,2,2-pentafluoroethyl))estra-4,9,15-trien-3-one;

17β-hydroxy-17α-(1,1,2,2,2-pentafluoroethyl)-11β-[4-(3-pyridinyl)phenyl]estra-4,9,15-trien-3-one;

6′-acetyl-9,11α-dihydro-17β-hydroxy-17α-(1,1,2,2,2-pentafluoroethyl)-4′H-naphth[3′,2′,1′:10,9,11]estr-4-en-3-one;

4-[9,11α-dihydro-17β-hydroxy-3-oxo-17α-(1,1,2,2,2-pentafluoroethyl)-4′H-naphth[3′,2′,1′:10,9,11)]estr-4-en-6′-yl]benzonitrile;

9,11α-dihydro-6′-(4-fluorophenyl)-17β-hydroxy-17α-(1,1,2,2,2-pentafluoroethyl)-4′H-naphth[3′,2′,1′:10,9,11]estr-4-en-3-one;

9,11α-dihydro-17β-hydroxy-17α-(1,1,2,2,2-pentafluoroethyl)-6′-(3-pyridinyl)-4′H-naphth[3′,2′,1′:10,9,11]estr-4-en-3-one;

6′-acetyl-9,11α-dihydro-17β-hydroxy-17α-(1,1,2,2,2-pentafluoroethyl)-4′H-naphth[3′,2′,1′:10,9,11]estra-4,15-dien-3-one;

4-[9,11α-dihydro-17β-hydroxy-3-oxo-17α-(1,1,2,2,2-pentafluoroethyl)-4′H-naphth[3′,2′,1′:10,9,11]estra-4,15-dien-6′-yl]benzonitrile;

9,11α-dihydro-6′-(4-fluorophenyl)-17β-hydroxy-17α-(1,1,2,2,2-pentafluoroethyl)-4′H-naphth[3′,2′,1′:10,9,11]estra-4,15-dien-3-one;

9,11α-dihydro-17β-hydroxy-17α-(1,1,2,2,2-pentafluoroethyl)-6′-(3-pyridinyl)-4′H-naphth[3′,2′,1′:10,9,11]estra-4,15-dien-3-one;

17β-hydroxy-11β-(4-hydroxyphenyl)-17α-(1,1,2,2,2-pentafluoroethyl)estra-4,9-dien-3-one;

17β-hydroxy-11β-(4-hydroxyphenyl)-17α-(1,1,2,2,2-pentafluoroethyl)estr-4-en-3-one;

9,11α-dihydro-6′,17β-dihydroxy-17α-(1,1,2,2,2-pentafluoroethyl)-4′H-naphth[3′,2′,1′:10,9,11]estr-4-en-3-one;

11β-[4-(acetyloxy)phenyl]-17β-hydroxy-17α-(1,1,2,2,2-pentafluoroethyl)estra-4,9-dien-3-one;

11β-[4-(acetyloxy)phenyl]-17β-hydroxy-17α-(1,1,2,2,2-pentafluoroethyl)estr-4-en-3-one

6′-acetyloxy-9,11α-dihydro-17β-hydroxy-17α-(1,1,2,2,2-pentafluoroethyl)-4′H-naphth[3′,2′,1′:10,9,11]estr-4-en-3-one;

17β-hydroxy-11β-[4-(hydroxymethyl)phenyl]-17α-(1,1,2,2,2-pentafluoroethyl)estra-4,9-dien-3-one;

17β-hydroxy-11β-[4-(hydroxymethyl)phenyl]-17α-(1,1,2,2,2-pentafluoroethyl)estr-4-en-3-one;

9,11α-dihydro-17β-hydroxy-6′-hydroxymethyl-17α-(1,1,2,2,2-pentafluoroethyl)-4′H-naphth[3′,2′,1′:10,9,11]estr-4-en-3-one;

4-[17β-hydroxy-3-oxo-17α-(1,1,2,2,2-pentafluoroethyl)estra-4,9-dien-11β-yl]benzaldehyde;

4-[17β-hydroxy-3-oxo-17α-(1,1,2,2,2-pentafluoroethyl)estr-4-en-11β-yl]benzaldehyde;

9,11α-dihydro-17β-hydroxy-3-oxo-17α-(1,1,2,2,2-pentafluoroethyl)-4′H-naphth[3′,2′,1′:10,9,11]estr-4-en-6′-al;

4-[17β-hydroxy-3-oxo-17α-(1,1,2,2,2-pentafluoroethyl)estra-4,9-dien-11β-yl]benzoic acid methyl ester;

4-[17β-hydroxy-3-oxo-17α-(1,1,2,2,2-pentafluoroethyl)estr-4-en-11β-yl]benzoic acid methyl ester;

9,11α-dihydro-17β-hydroxy-3-oxo-17α-(1,1,2,2,2-pentafluoroethyl)-4′H-naphth[3′,2′,1′:10,9,11]estr-4-en-6′-carboxylic acid methyl ester;

17β-hydroxy-11β-[4-(1-hydroxyethyl)phenyl]-17α-(1,1,2,2,2-pentafluoroethyl)estra-4,9-dien-3-one;

17β-hydroxy-11β-[4-(1-hydroxyethyl)phenyl]-17α-(1,1,2,2,2-pentafluoroethyl)estr-4-en-3-one;

9,11α-dihydro-17β-hydroxy-6′-(1-hydroxyethyl)-17α-(1,1,2,2,2-pentafluoroethyl)-4′H-naphth[3′,2′,1′:10,9,11]estr-4-en-3-one.

Preferred is 11β-(4-acetylphenyl)-17β-hydroxy-17α-(1,1,2,2,2-pentafluoroethyl)estra-4,9-dien-3-one (≡Compound A).

The antigestagens can, for example, be applied locally, topically, enterally or parenterally.

For the preferred oral administration, particularly suitable are tablets, dragees, capsules, pills, suspensions or solutions which can be prepared in a conventional manner with additives and carriers used in pharmacy. For local or topical application, vaginal pessaries or percutaneous systems such as skin plasters can be used for example. For parenteral application, particularly suitable are solutions, preferably oily or aqueous solutions, as well as suspensions or emulsions. Ampules are convenient unlit dosages.

According to a preferred mode of operation, the 17α-fluoralkylated progesterone receptor antagonist is typically administered over a period of 1 to 6 days, preferably 1 to 4 days, and more preferably on day(s). 1-3 after ovulation and/or the removal of oocytes. Typically, the antigestagen is administered over a period of 1 to 6 days, preferably 1-4 days, and more preferably 1-3 days after ovulation induction with e.g. a chorionic gonadotropin.

Surprisingly, according the invention, it has been found that low doses of a 17α-fluoralkylated progesterone receptor antagonists are effective in the methods disclosed herein. The 17α-fluoralkylated progesterone receptor antagonists are preferably administered to a human female subject in a daily dosage amount of up to 10 mg per subject, preferably 0.1-2 mg per subject, more preferably 0.1-1 mg per subject, and most preferably 0.1-0.7 mg per subject. For mammals in general, a daily dosage amount is typically 0.01-1 mg/kg, preferably 0.01-0.3 mg/kg, and more preferably 0.01-0.1 mg/kg. The daily dose of antigestagen can be administered as a single dose or as divided dosages throughout the day.

In a preferred embodiment according to the invention, the antigestagen is administered on a single day to a human subject in an amount of 0.1-2 mg/per subject, more preferably 0.1-1 mg/per subject, and most preferably 0.1-0.7 mg/per subject or to a mammal in an amount of 0.01-1 mg/kg, preferably 0.01-0.3 mg/kg, and more preferably 0.01-0.1 mg/kg. For example, on day 1, 2, 3, 4, 5 or 6 following ovulation, removal of oocytes, or administration of a chorionic gonadotropin, preferably day 1-3, and more preferably day 2.

For any particular 17α-fluoralkylated progesterone receptor antagonist, the most appropriate dose can be determined, for example, by evaluation of the potency to induce premature menstruation in advanced luteal phase of the human cycle as described in e.g. Herrrmann, W., et al, 1982, Comptes Rendus 294:933. The use of antigestagens for delaying endometrial maturation has been previously described, for example, in U.S. Pat. No. 4,764,513, EP 0219447B1, Hegele-Hartung et al., 1992, Endocrinology 131:2446-2460, all of which are incorporated herein by reference in their entirety.

The compounds of this invention can be employed in admixture with conventional excipients, i.e., pharmaceutically acceptable organic or inorganic carrier substances suitable for, for example, parenteral, enthral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylase or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined, where desired, with other active agents, e.g., vitamins.

Sustained or directed release compositions can be formulated, e.g., liposomes or those wherein the active compound is protected with differentially degradable coatings, e.g., by microencapsulation, multiple coatings, etc. It is also possible to freeze-dry the new compounds and use the lyophilizates obtained, for example, for the preparation of products for injection.

For topical application, there are employed as nonsprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds of a known agent, e.g., by means of an appropriate, conventional pharmacological protocol.

### BRIEF DESCRIPTION OF THE FIGURES

FIG. 1 is a schematic illustration of the experiment described in Example 1. The day of administration of human chorionic gonadotropin (hCG) was designated as day 0 of pseudopregnancy (d0). As shown in the diagram, ovarian stimulation of the animals occurred on −d3, −d2 and −d1 of pseudpregnancy induction and the progesterone receptor antagonist was administered on d2 of psuedopregnancy.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosure of all applications, patents and publications, cited above or below, are hereby incorporated by reference.

### EXAMPLES

### Example 1

Improvement of Endometrial Receptivity with Low Doses of a 17α-Fluoralkylated Progesterone Receptor Antagonist in the Superovulated Rabbit Model.

The effects of a 17α-fluoralkylated progesterone receptor antagonist on endometrial activity was evaluated in the superovulated pseudopregnant rabbit model. The pseudopregnancy of the rabbit has proven to be a model system for the human luteal phase (Beier and Kuhnel, 1973, Hormone Res. 4:1-27; Fischer et al., 1985, Fertilitat 1:101-109. In this species, ovarian stimulation induces an advancement of secretory transformation (Delbos-Winter et al., 1987, Fertilitat 3: 87-93) as well as an enhancement of gland formation.

A total of 25 sexually mature nulliparous New Zealand White rabbits with a body weight of 3.1 to 4.0 kg were used for the analysis. The animals were divided into two control groups (groups 1 and 2) and three treatment groups (groups 3-5), with five animals in each group.

Control group 1 received 75 international units (IU) of human chorionic gonadotropin (hCG) administered intravenously in order to induce pseudopregnancy. The day of hCG-injection was designated as day 0 of pseudopregnancy (d0 p.hCG).

Control group 2 and treatment groups 3, 4, and 5 were gonadotropin-stimulated with 5 IU of the human menopausal gonadotropin (HMG) Pergonal® (Serono Pharma, Unterschleipheim, Germany) for 3 days, by subcutaneous injection, in order to induce multiple follicular growth. Pseudopregnancy was induced by the administration of human chorionic gonadotropin (hCG) as described for control group 1 above.

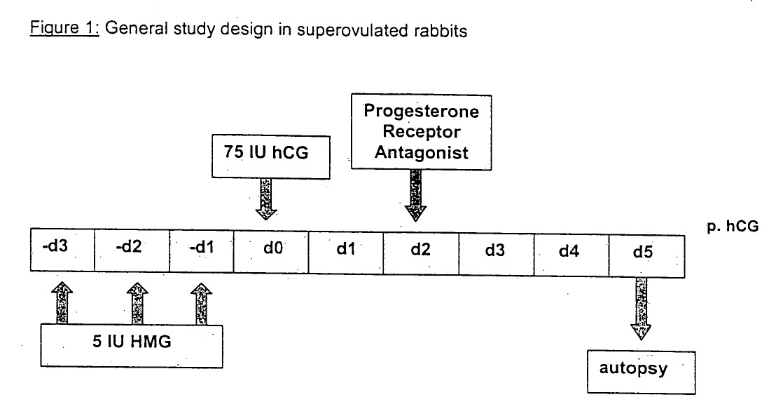

Treatment groups 3, 4 and 5 also received a single per oral application of a 17α-fluoralkylated progesterone receptor antagonist (Compound A) two days after pseudopregnancy induction with the administration of human chorionic gonadotropin (d2 p.hCG). The detailed treatment schedule is shown in FIG. 1.

Animals in all treatment groups were sacrificed on day 5 following pseudopregnancy induction (d5 p.hCG), and uteri were removed. Relative uterine wet weights were determined (in mg/100 g body weight) and a part of the uterus was snap frozen in liquid nitrogen for determination of uteroglobin expression by in situ hybridization. Uteroglobin expression is known as a highly sensitive parameter for determination of endometrial receptivity (Beier, H. M., 1968, Biochem Biophys. Acta. 160:289-291; Beier, H. M., 1982, in: Beier, H. M. and Karlson, P. (eds.), Proteins and Steroids in Early Pregnancy), and is a marker for secretory activity (Beier, 1976, J. Reprod. Fertil. Suppl. 25:53-69) and differentiation of the endometrial epithelium (Krusche & Beier, 1994, Ann. Anat. 176:23-31). In situ hybridization for detection of uteroglobin expression was conducted as previously described in Krusche & Beier, above. Another piece of the uterus was removed and processed for paraffin histology in order to determine the endometrial transformation status using the McPhail-Index (McPhail M k, 1934, J. Phys. 83:145-156) The McPhail Index reflects different degrees of progestogenic (transforming) activity in the rabbit endometrium:

1. no transformation

2. low transformation

3. pronounced transformation

4. high transformation

The results are presented in Table 1 below.

TABLE 1
Effect of a single treatment of a 17α-fluoralkylated progesterone receptor
antagonist on uterus weight, endometrial transformation, and uteroglobin
mRNA expression at five days after pseudopregnancy induction
(d5 p.hCG) in the superovulated pseudopregnant rabbit.

| | | | | Measurement endpoints | | |
|---|---|---|---|---|---|---|
| | Treatment Schedule | | | at d5 p. hCG | | |
| | | | d2 | | | Uteroglobin |
| | | | p.hCG | Uterine | | ISH |
| | −d3, −d2, −d1 | d0 | Progesterone | wet | | [expression |
| | p.hCG | p.hCG | receptor | weight | | in luminal |
| | 5 IU | 75 IU | antagonist | [mg/kg] | Mc-Phail | epithelial |
| Group | HMG | HCG | [mg/kg] | [x ± SD] | [x ± SD] | cells] |
| 1 | − | + | — | 324 ± 99 | 3.2 ± 0.2 | very high |
| | | | | | | expression |
| 2 | + | + | 0 | 448 ± 80 | 3.9 ± 0.1 | very low |
| | | | | | | expression |
| 3 | + | + | 0.1 | 305 ± 48 | 3.0 ± 0.1 | very high |
| | | | | | | expression |
| 4 | + | + | 1 | 227 ± 56 | 1.5 ± 0.3 | very low |
| | | | | | | expression |
| 5 | + | + | 10 | 144 ± 30 | 1.1 ± 0.3 | absent |

As shown in the table, animals which were subject to ovarian hyperstimulation, but which were not treated with an antigestagen (Group 2), showed advanced endometrial maturation as demonstrated by nearly indetectable uteroglobin expression in endometrial luminal epithelial cells, the weight of the uterus, and the relatively high Mc-Phail index score. However, animals which were subject to ovarian hyperstimulation and which were treated with an antigestagen, Groups 3-5, showed a delay in endometrial maturation, as demonstrated by the uterine wet weights and the Mc-Phail index scores. For animals which were treated with a low dose of progesterone receptor antagonist, 0.1 mg/kg (Group 3), endometrial maturation roughly corresponded with that of control animals which were not subject to ovarian hyperstimulation (Group 1). In contrast, higher doses of progesterone receptor antagonist, 1 mg/kg (group 4) and 10 mg/kg (group 5), apparently counteracted the advancement of endometrium maturation associated with ovarian hyperstimulation and also delayed the timing of maturation beyond that observed in animals which were not subject to hyperovarian stimulation (Group 1).

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of inhibiting the occurrence of advanced endometrium maturation in a human female subject undergoing fertility enhancing treatment comprising
administering at least one 17α-fluoralkylated progesterone receptor antagonist to the female subject during the post-ovulatory phase of the endometrial cycle.

2. A method according to claim 1, wherein the 17α-fluoralkylated progesterone receptor antagonist is administered to the subject in a daily dosage amount of 0.1-2 mg per subject.

3. A method according to claim 2, wherein the fertility treatment comprises the administration to the subject of a follicle stimulating agent comprising follicle stimulating hormone.

4. A method according to claim 2, wherein the 17α-fluoralkylated progesterone receptor antagonist is administered in an amount of 0.1-2 mg per subject on a single day during the post-ovulatory phase of the endometrial cycle.

5. A method according to claim 2, wherein the 17α-fluoralkylated progesterone receptor antagonist is a compound of formula I: 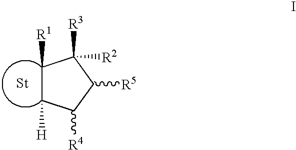
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 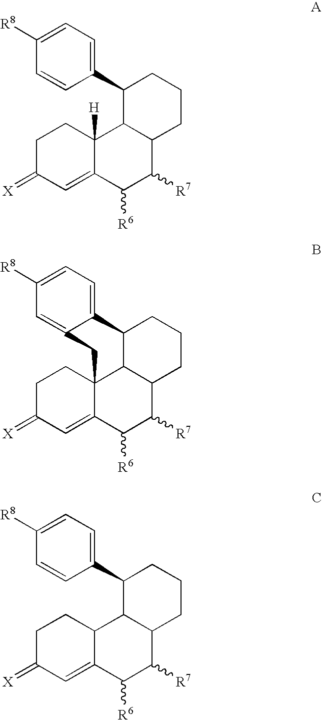
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

6. A method according to claim 4, wherein the 17α-fluoralkylated progesterone receptor antagonist is administered orally to the subject.

7. A method of achieving pregnancy in a human female subject comprising
stimulating the ovaries of the subject by administering a follicle stimulating agent to the subject, wherein the agent comprises follicle stimulating hormone;
removing eggs from the ovary of the stimulated subject;
administering at least one 17α-fluoralkylated progesterone receptor antagonist to the subject in the post-ovulatory phase of the endometrial cycle;
fertilizing at least one egg in vitro to obtain an embryo;
transferring the embryo into the uterus or fallopian tubes of the mammal.

8. A method according to claim 7, wherein the 17α-fluoralkylated progesterone receptor antagonist is administered to the subject in a daily dosage amount of 0.1-10 mg per subject

9. A method according to claim 8, wherein the 17α-fluoralkylated progesterone receptor antagonist is administered in an amount of 0.1-2 mg per subject on a single day during the post-ovulatory phase of the endometrial cycle.

10. A method according to claim 8, wherein the 17α-fluoralkylated progesterone receptor antagonist is a compound of formula I: 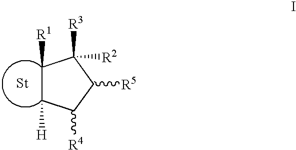
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 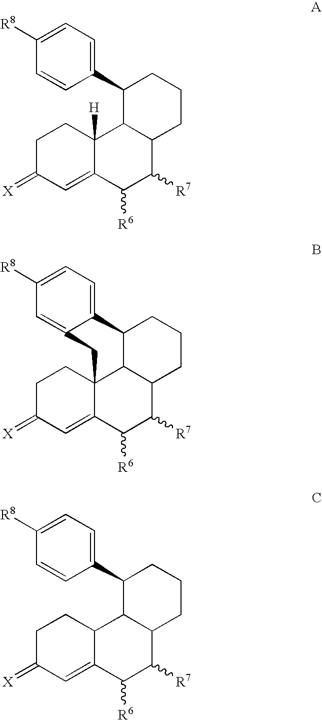
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

11. A method according to claim 9, wherein the 17α-fluoralkylated progesterone receptor antagonist is administered orally to the subject.

12. A method of inhibiting the occurrence of advanced endometrium maturation in a non-human female mammal undergoing fertility enhancement treatment to achieve pregnancy comprising
administering at least one 17α-fluoralkylated progesterone receptor antagonist to the mammal during the post-ovulatory phase of the endometrial cycle.

13. A method according to claim 12, wherein the 17α-fluoralkylated progesterone receptor antagonist is administered to the mammal in a daily dosage amount of 0.01-1 mg/kg.

14. A method according to claim 13, wherein the fertility treatment comprises the administration to the mammal of a follicle stimulating agent comprising follicle stimulating hormone.

15. A method according to claim 13, wherein the 17α-fluoralkylated progesterone receptor antagonist is administered to the mammal in an amount of 0.1-1 mg/kg on a single day during the post-ovulatory phase of the endometrial cycle.

16. A method according to claim 13, wherein the 17α-fluoralkylated progesterone receptor antagonist is a compound of formula I: 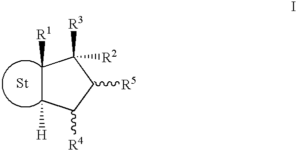
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 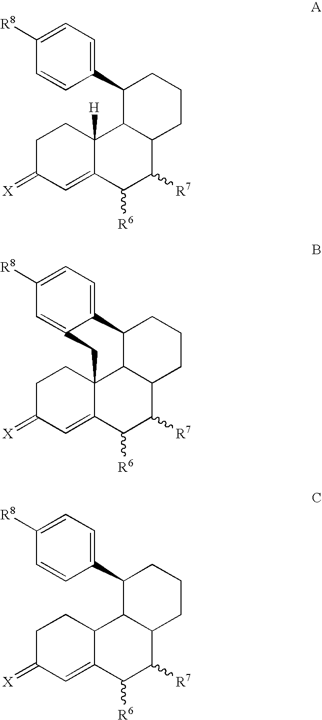
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

17. A method of achieving pregnancy in a non-human mammal comprising
stimulating the ovaries of the mammal by administering a follicle stimulating agent to the mammal, wherein the agent comprises follicle stimulating hormone;
removing eggs from the ovary of the stimulated mammal;
administering at least one 17α-fluoralkylated progesterone receptor antagonist to the mammal in the post-ovulatory phase of the endometrial cycle;
fertilizing at least one egg in vitro to obtain an embryo;
transferring the embryo into the uterus or fallopian tubes of the mammal.

18. A method according to claim 17, wherein the 17α-fluoralkylated progesterone receptor antagonist is administered to the mammal in a daily dosage amount of 0.01-1 mg/kg.

19. A method according to claim 18, wherein the 17α-fluoralkylated progesterone receptor antagonist is administered to the mammal in an amount 0.1-1 mg/kg on a single day during the post-ovulatory phase of the endometrial cycle.

20. A method according to claim 18, wherein the 17α-fluoralkylated progesterone receptor antagonist is a compound of formula I: 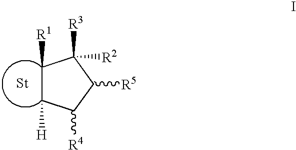
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 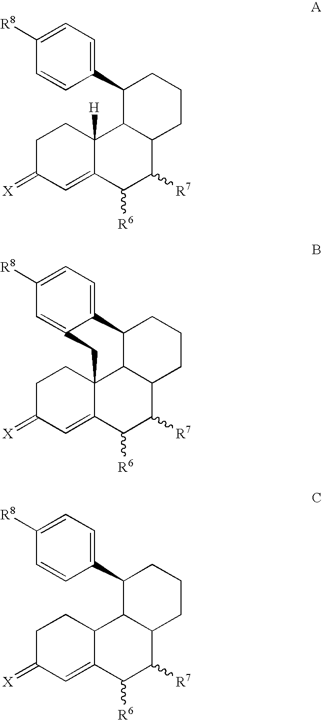
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

21. A non-human mammal which results from a pregnancy achieved by a process according to claim 13.

22. A non-human mammal which results from a pregnancy achieved by a process according to claim 18.

23. A method of inhibiting the occurrence of advanced endometrium maturation in a human female subject undergoing fertility enhancing treatment comprising
administering at least one compound of formula I to the subject, wherein formula I is 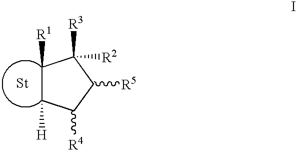
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 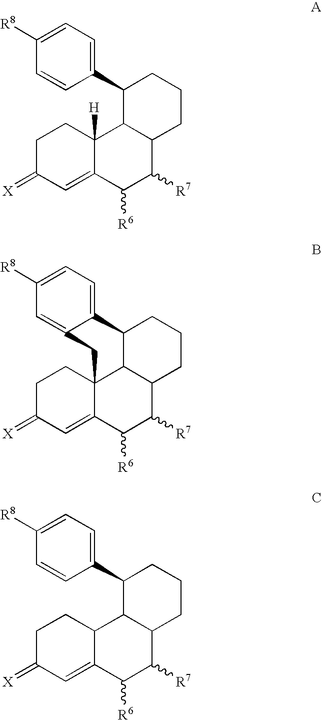
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

24. A method of achieving pregnancy in a human female subject comprising
stimulating the ovaries of the subject by administering a follicle stimulating agent to the subject, wherein the agent comprises follicle stimulating hormone;
removing eggs from the ovary of the stimulated subject;
administering at least one compound of formula I to the subject in the post-ovulatory phase of the endometrial cycle;
fertilizing at least one egg in vitro to obtain an embryo;
transferring the embryo into the uterus or fallopian tubes of the mammal, wherein formula I is 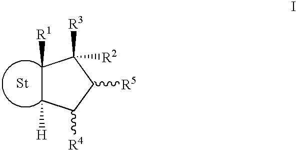
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 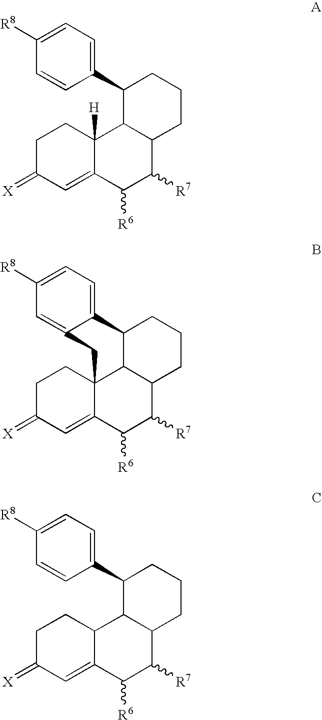
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

25. A method of inhibiting the occurrence of advanced endometrium maturation in a non-human female mammal undergoing fertility enhancement treatment to achieve pregnancy comprising
administering at least one compound according to formula I to the mammal, wherein formula I is 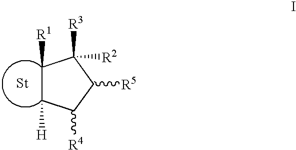
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 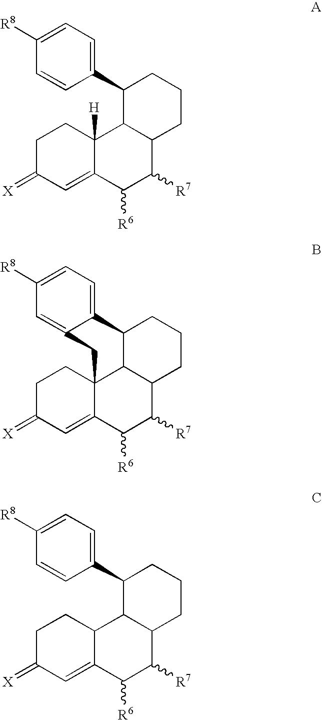
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

26. A method of achieving pregnancy in a non-human mammal comprising
stimulating the ovaries of the mammal by administering a follicle stimulating agent to the mammal, wherein the agent comprises follicle stimulating hormone;
removing eggs from the ovary of the stimulated mammal;
administering at least one compound of formula I to the mammal in the post-ovulatory phase of the endometrial cycle;
fertilizing at least one egg in vitro to obtain an embryo;
transferring the embryo into the uterus or fallopian tubes of the mammal,
wherein formula I is 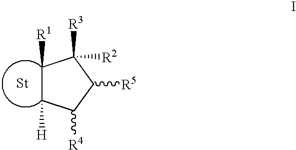
wherein
R¹ is methyl or ethyl,
R² is CₙFₘHₒ, wherein n is 1-6, preferably 2, 3, 4, 5 or 6, m>1 and m+o=2n+1,
R³ is a free, etherified or esterified hydroxy group,
R⁴ and R⁵ each is a hydrogen, or together form an additional bond or a methylene group,
St is a steroidal ABC-ring system of partial formula A, B or C 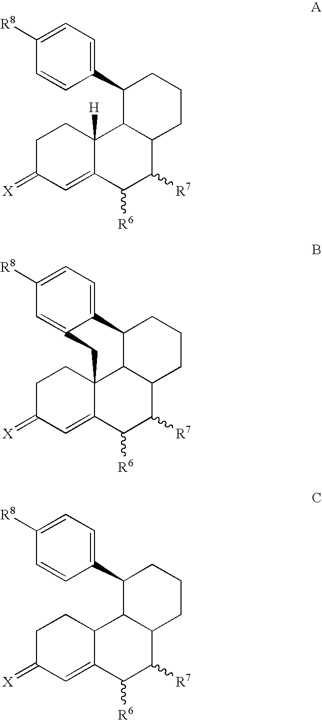
wherein
R⁶ is hydrogen, a straight-chain C₁-C₄ alkyl group or branched C₃-C₄ alkyl group or halogen,
R⁷ is hydrogen, a straight-chain C₁-C₄ alkyl group or a branched C₃-C₄ alkyl group, or
if St is a steroidal ABC-ring system A or B, in addition
R⁶ and R⁷ together can form an additional bond,
X is oxygen, hydroxyimino (═N—OH) or two hydrogen atoms,
R⁸ is Y or aryl that is optionally substituted in several places with a group Y, other than H,
Y is hydrogen, halogen, —OH, —NO₂, —N₃, —CN, —NR^{9a}R^{9b}, —NHSO₂R⁹, —CO₂R⁹, C₁-C₁₀ alkyl, C₁-C₁₀ alkoxy, C₁-C₁₀ alkanoyloxy, benzoyloxy, C₁-C₁₀ alkanoyl, C₁-C₁₀ hydroxyalkyl or benzoyl,
R^{9a} and R^{9b} are the same or different and each is hydrogen or C₁-C₁₀ alkyl,
R⁹ is hydrogen or C₁-C₁₀ alkyl,
and for —NR^{9a}R^{9b} radicals, as well as their physiologically compatible salts with acids and for —CO₂R⁹ radicals with R⁹ being hydrogen, as well as their physiologically compatible salts with bases.

27. A method of inhibiting the occurrence of advanced endometrium maturation in a human female subject undergoing fertility enhancing treatment comprising
administering at least one 17α-fluoralkylated progesterone receptor antagonist to the female subject during the post-ovulatory phase of the endometrial cycle.

28. A method of inhibiting the occurrence of advanced endometrium maturation in a human female subject undergoing fertility enhancing treatment comprising
administering at least one 17α-fluoralkylated progesterone receptor antagonist to the female subject during the post-ovulatory phase of the endometrial cycle after said fertility enhancing treatment.

29. A method of inhibiting the occurrence of advanced endometrium maturation in a non-human female mammal undergoing fertility enhancement treatment to achieve pregnancy comprising
administering at least one 17α-fluoralkylated progesterone receptor antagonist to the mammal during the post-ovulatory phase of the endometrial cycle after said fertility enhancing treatment.
